(19) United States
(12) Patent Application Publication
(10) Pub. No.: US 2015/0173642 A1
(43) Pub. Date: Jun. 25, 2015

(54) METHOD AND APPARATUS FOR MAGNETIC RESONANCE IMAGING

(71) Applicant: Siemens Aktiengesellschaft, Muenchen (DE)

(72) Inventors: Jan Ole Blumhagen, Erlangen (DE); Dominik Paul, Bubenreuth (DE)

(73) Assignee: Siemens Aktiengesellschaft, Muenchen (DE)

(21) Appl. No.: 14/576,650

(22) Filed: Dec. 19, 2014

(30) Foreign Application Priority Data
Dec. 19, 2013 (DE) 102013226638.3

(51) Int. Cl. A61B 5/055; A61B 5/113; A61B 5/00; A61B 5/0402

(52) U.S. Cl. CPC A61B 5/055; A61B 5/0402; A61B 5/1135; A61B 5/7285

(57) ABSTRACT

In a method and apparatus for magnetic resonance imaging, an improved saturation of magnetic resonance signals during an acquisition sequence is achieved by the acquisition sequence including at least one acquisition cycle, this acquisition cycle including a saturation pulse set composed of one or more saturation pulses, a first trigger window and a second trigger window. The first trigger window and the second trigger window are temporally delimited from one another. The first trigger window and the second trigger window are activated on the basis of a trigger signal. At least one saturation pulse of the saturation pulse set takes place during the first trigger window. Data acquisition takes place during the second trigger window.

### BACKGROUND OF THE INVENTION

1. Field of the Invention

The invention concerns a method for magnetic resonance imaging, a magnetic resonance apparatus and a non-transitory, computer-readable data storage medium encoded with programming instructions for implementing such a method.

2. Description of the Prior Art

In magnetic resonance imaging, the acquisition of magnetic resonance image data of an examination subject by operation of a magnetic resonance apparatus is controlled using acquisition sequences (magnetic resonance sequences). Acquisition sequences often produce a saturation of magnetic resonance signals of specific tissue types. In the magnetic resonance image data, the saturation typically causes suppression of the magnetic resonance signals emanating from the specific tissue types. For example, many acquisition sequences provide a fat saturation that can be used to improve the contrast between fat tissue and other tissue types. Alternatively, fat saturation can also be used to emphasize fat tissue in the image.

Furthermore, triggered acquisition sequences are used in magnetic resonance imaging that provide triggering of the data acquisition of the magnetic resonance signals, for example using an external trigger signal. Particularly in triggered acquisition sequences, an incomplete saturation of the magnetic resonance signals can occur.

### SUMMARY OF THE INVENTION

An object of the invention is to provide a method for magnetic resonance imaging of an examination subject using an acquisition sequence that includes at least one acquisition cycle, wherein the acquisition cycle includes a saturation pulse set with one or more saturation pulses, a first trigger window and a second trigger window, wherein
the first trigger window and the second trigger window are temporally delimited from one another,
the first trigger window and the second trigger window are activated on the basis of a trigger signal,
at least one saturation pulse of the saturation pulse set takes place during the first trigger window, and
a data acquisition takes place during the second trigger window.

Then examination subject can be a patient, a training person or a phantom. The acquisition sequence is typically used by a magnetic resonance apparatus. An acquisition sequence is typically a pulse sequence. An acquisition cycle can include a sequence of saturation pulses and a data acquisition which is repeated cyclically within the acquisition sequence. An acquisition cycle can be a cycle of the change of the trigger signal, such as a cyclical change. An acquisition cycle can be a breathing cycle and/or a cardiac cycle of the examination subject. Different slices and/or different portions of k-space are typically acquired in different acquisition cycles. In the acquisition sequence, the acquisition cycles can be repeated until all predetermined k-space lines and/or all predetermined slices of the magnetic resonance image are acquired.

A saturation pulse can have the effect of causing a value of a magnetization (for example the longitudinal magnetization) to go substantially to zero in an examination volume. A saturation pulse is typically tissue-specific, which means that the saturation pulse largely sets to zero only the magnetization of a specific tissue type. Saturation pulses can thus select the type of tissue from which magnetic resonance signals can be acquired. Saturation pulses can be fat saturation pulses, which means that the magnetization (in particular the longitudinal magnetization) of fat tissue is set to zero (saturated). After application of a saturation pulse, only a transverse magnetization (in particular for the specific tissue type) typically still exists. For this purpose, a saturation pulse can include a spoiler gradient to dephase the magnetization. A saturation pulse thus typically largely erases the history in the magnetization, in particular of the longitudinal magnetization, since the saturation pulse typically sets the magnetization to zero without consideration of the preceding values of the magnetization. A saturation pulse thus is typically non-selective. Therefore, a saturation pulse typically acts over at least a partial region of an acquisition volume, in particular over the entire acquisition volume. A saturation pulse conventionally acts independently of movement (in particular a breathing movement) of the examination subject.

The data acquisition typically includes at least one readout window that includes the activation of a receiver for the magnetic resonance signals, for example of an ADC (analog/digital converter) that is coupled to reception coils of the magnetic resonance apparatus. The data acquisition furthermore typically includes an excitation pulse to excite the magnetization in the measurement volume. An excitation pulse typically ensures that a magnetic resonance signal can be read from an examination region. The data acquisition furthermore may include at least one refocusing pulse to refocus the magnetization in the measurement volume. The excitation pulse typically takes place at the start of the data acquisition. The refocusing pulses and readout windows then typically take place in alternation after an excitation of the magnetization has taken place by means of the excitation pulse. The data acquisition during an acquisition cycle can include an entire echo train within the scope of a turbo spin echo acquisition. During the data acquisition, one or more k-space lines of one or more slices of a magnetic resonance image are typically acquired (filled with data). The data acquisition does not include the acquisition of the trigger signal.

The first trigger window and second trigger window are typically time windows within the acquisition cycle that are activated on the basis of the trigger signal. The first trigger window and second trigger window can fill the entire acquisition cycle. Alternatively, the acquisition cycle can include additional time windows during which in particular neither a data acquisition nor a saturation by means of saturation pulses (in particular a measurement pause) takes place. The first trigger window and/or second trigger window is typically activated depending on signal states of the trigger signal and/or of a phase of the (in particular cyclical) change of the trigger signal. For example, the first trigger window and/or the second trigger window can be activated on the basis of a cyclical movement of the examination subject, for example due to breathing states and/or cardiac phases of the examination subject. The first trigger window and the second trigger window should be activated given different signal states of the trigger signal. For the activation of the first trigger window, a trigger state that is separate from the activation of the second trigger window is used. The second trigger window should thus not simply follow the first trigger window due to a time relation.

The second trigger window can be matched to the acquisition cycle such that the data acquisition begins at defined breathing states of the examination subject. For the activation of the second trigger window the presence of less movement of the examination subject is thereby advantageous, for example a flat breathing, in particular only a slight breathing movement or no breathing movement, for example during the exhalation or inhalation. No data acquisition should take place during the first trigger window. A saturation pulse of the saturation pulse set can also be present during the second trigger window. The first trigger window and/or the second trigger window can also include a gating of the acquired magnetic resonance signals.

The procedure disclosed herein is based on the consideration that an incomplete saturation of the tissue signals (in particular of the fat tissue) is often present given conventional triggered acquisition sequences. This is typically expressed such that the slices of the magnetic resonance images that are acquired immediately after the triggering of the data acquisition have a lower saturation of the tissue signals than the slices of the magnetic resonance images that are acquired later during the acquisition cycle. This leads to unwanted inhomogeneities in the tissue depiction in the magnetic resonance images. The reason for this inhomogeneous saturation of the tissue signals typically lies in an incomplete saturation of the tissue signals at the beginning of the data acquisition of an acquisition cycle. This is in turn due to the fact that, given conventionally triggered acquisition sequences, the saturation pulses only take place together with the start of the data acquisition, i.e. exclusively during the second trigger window (in particular at the beginning of the second trigger window).

A saturation of the tissue signals typically becomes sufficient only after the application of multiple saturation pulses, since only then is the steady state necessary for sufficient saturation of the tissue signals present. Since (in particular temporally varying) pauses between the respective data acquisitions are present relative to conventional acquisition sequences due to the triggering of the data acquisition, the application of the saturation pulses is interrupted for a respectively longer period of time in conventional acquisition sequences, and the steady state that is necessary for sufficient saturation of tissue signals no longer exists at the beginning of a data acquisition. Given conventionally triggered acquisition sequences, the tissue signal to be suppressed is then relaxed back again due to the interruption of the application of the saturation pulses and the interruption of the steady state that follows this, and therefore said tissue signal is no longer completely saturated. The incomplete saturation of the tissue signals in the magnetic resonance images therefore results, and thus the reduced image quality of the magnetic resonance images acquired by conventional acquisition sequences.

The fact that the acquisition sequence according to the invention includes a first trigger window in addition to the second window, wherein at least one saturation pulse of the saturation pulse set takes place during the first trigger window, advantageously leads to a saturation of the tissue signals that is improved relative to conventional acquisition sequences. The first trigger window, and thus the at least one saturation pulse of the saturation pulse set, advantageously take place within the acquisition sequence chronologically before the second trigger window (and thus the data acquisition). A pre-saturation of the tissue signals thus advantageously takes place during a pre-saturation phase during the first trigger window, before the data acquisition by the at least one saturation pulse that takes place during the first trigger window. Like the data acquisition, the pre-saturation advantageously takes place due to a triggering by the trigger signal (in particular a triggering that is separate from the data acquisition) so that the pre-saturation takes place so as to be matched chronologically with the data acquisition. The at least one saturation pulse of the saturation pulse set that takes place during the first trigger window thereby takes place in addition to possible saturation pulses of the saturation pulse set that, in conventional acquisition sequences, take place during the second trigger window at the beginning of the data acquisition.

Since saturation pulses are typically non-selective and thus are not sensitive to movement, the saturation pulses can take place during the first trigger window during which a more significant movement of the examination subject is typically present. The data acquisition (which is sensitive to movements of the examination subject) then takes place during the second trigger window (during which less movement of the examination subject is present). The first trigger window is thus advantageously placed in a time period during which the movement of the examination subject is more significant than during the second trigger window. The movement phase of the examination subject, which is disadvantageous for the data acquisition during the second trigger window, can thus be utilized for the saturation of the tissue signals during the first trigger window.

At the beginning of the data acquisition, the at least one saturation pulse therefore already leads to a pre-saturation of the tissue signals and an adjustment and/or maintenance of the steady state that is necessary for sufficient saturation of the tissue signals. The magnetic resonance images acquired using such an acquisition sequence thus have a more homogeneous (in particular complete) saturation of the tissue signals relative to magnetic resonance images acquired by means of conventional acquisition sequences, in particular across all slices of the magnetic resonance images. An extension of the measurement time thus is not necessary.

In an embodiment, the trigger signal is a physiological signal of the examination subject and/or an external trigger signal. For example, trigger signals are generated by means of a physiological signal measured during the implementation of the acquisition sequence. For example, the physiological signal can describe breathing movement or a heartbeat of the examination subject, in particular of an examined person. The physiological signal can be measured by additional devices, for example by an electrocardiograph or a breathing belt. The physiological signal can also be measured by the magnetic resonance apparatus. For example, magnetic resonance navigator sequences can be implemented for magnetic resonance tomography, and thus movement of the examination subject can be detected (for example of the diaphragm of the examination subject). In particular, prominent points in the signal curve of the physiological signals can be used to trigger the first trigger window and/or second trigger window. This can be the case when the breathing belt and/or the magnetic resonance navigator sequence indicates a specific breathing position of the examination subject. Trigger signals can also supply gating information that establishes special time periods of the acquisition sequence, wherein only the measurement data acquired from these special time periods are used for the reconstruction of the magnetic resonance images. For example, the external trigger signal can be a synchronization signal and/or a signal predetermined by the user of the magnetic resonance apparatus.

In another embodiment, the first trigger window is activated depending on the position of the trigger signal in relation to at least one threshold. The at least one threshold is thereby typically used with regard to measured signal values of the trigger signal. The at least one threshold can thus establish a defined breathing state of the examination subject, for example. For example, for this purpose the breathing curve can be normalized to a maximum (in particular an averaged or absolute maximum), wherein then the at least one threshold is established for percentile proportions of the maximum of the breathing curve. Two thresholds are preferably used for the activation of the first trigger window. The first threshold can establish an activation of the first trigger window, in particular a beginning of the first trigger window. The second threshold can establish a deactivation of the first trigger window, in particular an end of the first trigger window. For example, the first threshold can thereby be situated in a breathing state of the examination subject which has a lower proportion of the maximum of the breathing curve than the second threshold. The first threshold can thus be a lower threshold of the physiological signal, while the second threshold is an upper threshold of the physiological signal. The adjustment of the at least one threshold for activation of the first trigger window is advantageously implemented such that the first trigger window is activated when a saturation of the tissue signals by means of the at least one saturation pulse is particularly advantageous for a following data acquisition. An improved saturation of the tissue signals can thus be achieved during the data acquisition.

In another embodiment, a learning phase is implemented to determine a pattern of the trigger signal, wherein the at least one threshold is determined on the basis of the pattern of the trigger signal. For example, one possible pattern of a trigger signal is the distance between points in an electrocardiogram, in particular the distance between two respective, successive R-spikes. An additional possible pattern of a trigger signal is a waveform (in particular a frequency of the waveform) of a breathing signal acquired by means of a breathing belt. A pattern can be determined just as well in the signals generated by means of the magnetic resonance navigator sequences. The pattern can depict a representation of how the physiological signals vary in the course of time. The pattern can also offer a depiction of the (in particular chronological) sequence of the trigger signal. The learning phase to determine the pattern of the trigger signal is preferably implemented at the beginning of the acquisition sequence and/or before the beginning of the acquisition sequence. For example, the pattern of the trigger signal can be determined using the first measured breathing cycles of the examination subject. For example, the further course of the breathing of the examination subject can thereby be extrapolated and it can be established when a suitable breathing state exists for the first trigger window. The at least one threshold can be implemented on the basis of a learning phase to determine an additional threshold for the second trigger window (i.e. for the data acquisition).

In a further embodiment, the at least one threshold is chosen such that the duration of the first trigger window has a minimum value which is required for at least one saturation pulse of the saturation pulse set. The duration of first trigger window therefore preferably amounts to more than one millisecond. If multiple such saturation pulses should be applied during the first trigger window, the duration of the first trigger window is advantageously adapted to the number of saturation pulses. Between two and four (preferably at most three) saturation pulses advantageously take place during the first trigger window of an acquisition cycle. An optimal saturation of the tissue signals for a following data acquisition can therefore be achieved. At the same time, the examination subject is not unnecessarily exposed to electromagnetic radiation (in particular due to too high a number of saturation pulses), such that an unnecessary heating of the examination subject can be avoided and the specific absorption rate (SAR) can be kept low. An advantageous duration of the first trigger window for two to four saturation pulses is accordingly between 20 and 100 milliseconds, preferably between 40 and 80 milliseconds.

In another embodiment the second trigger window essentially follows immediately after the first trigger window within the acquisition cycle. “Immediately” here means in particular that no additional trigger window and/or time window is switched between the first and second trigger window. “Immediately” can also mean that the end of the first trigger window represents the beginning of the second trigger window. For this, the second threshold of the trigger signal (which represents the end of the first trigger window) can advantageously represent an additional threshold for activation (in particular for the beginning) of the second trigger window. If the acquisition sequence and/or the triggering requires it, a short time window can also be present between the first trigger window and the second trigger window, which are activated separately from one another on the basis of the trigger signal. The at least one saturation pulse that takes place during the first trigger window cam enable an optimal saturation of the tissue signals for the data acquisition in the second trigger window, which data acquisition essentially follows immediately.

The magnetic resonance apparatus according to the invention has a control device, wherein the control device is designed to execute a method according to the invention. With the control device, the magnetic resonance apparatus can thus execute a method for magnetic resonance imaging of an examination subject using an acquisition sequence that includes at least one acquisition cycle. For this, the control device has a saturation pulse generator which is designed to generate a saturation pulse set with one or more saturation pulses. Furthermore, the control device has a trigger module that is designed to activate a first trigger window and a second trigger window on the basis of a trigger signal, wherein the first trigger window and the second trigger window are temporally delimited from one another. Furthermore, the magnetic resonance apparatus has a data acquisition device which is designed for data acquisition. The saturation pulse generator, the trigger module and the data acquisition device are matched to one another such that at least one saturation pulse of the saturation pulse set takes place during the first trigger window, and during the second trigger window a data acquisition takes place by operation of the data acquisition device.

According to another embodiment, the saturation pulse generator, the trigger module and the data acquisition device are matched to one another such that the trigger signal is a physiological signal of the examination subject and/or is an external signal.

According to another embodiment, the saturation pulse generator, the trigger module and the data acquisition device are matched to one another such that the first trigger window is activated depending on the position of the trigger signal in relation to at least one threshold.

According to another embodiment, the saturation pulse generator, the trigger module and the data acquisition device are matched to one another such that a learning phase is implemented to determine a pattern of the trigger signal, wherein the at least one threshold is determined on the basis of the pattern of the trigger signal.

According to another embodiment, the saturation pulse generator, the trigger module and the data acquisition device are matched to one another such that the at least one threshold is selected such that the duration of the first trigger window has a minimum value which is required for at least one saturation pulse of the saturation pulse set.

According to another embodiment, the saturation pulse generator, the trigger module and the data acquisition device are matched to one another such that the second trigger window chronologically follows immediately after the first trigger window within the acquisition cycle.

The control device can have additional control components that are necessary and/or advantageous for execution of a method according to the invention. The control device can also be designed to send control signals to the magnetic resonance apparatus and/or to receive and/or process control signals in order to execute a method according to the invention. Computer programs and additional software by means of which a processor of the control device automatically controls and/or executes a method workflow of a method according to the invention can be stored in a memory unit of the control device. The control device can be integrated into the magnetic resonance apparatus. The control device can also be installed separately from the magnetic resonance apparatus. The control device can be connected with the magnetic resonance apparatus. The magnetic resonance apparatus according to the invention thus enables an acquisition of magnetic resonance images by means of a triggered acquisition sequence, wherein the magnetic resonance images have a particularly homogeneous saturation of tissue signals, and thus a high image quality.

The storage medium according to the invention can be loaded directly into a memory of a programmable control device of a magnetic resonance apparatus and has program code in order to execute a method according to the invention when executed in the control device of the magnetic resonance apparatus. The method according to the invention thus can be executed quickly so as to be identically repeatable and robust. The program code causes the method steps according to the invention to be executed the control device. The control device must include the requirements (for example an appropriate working memory, a graphics card or a logic unit) so that the respective method steps can be executed efficiently. Examples of electronically readable data media are a DVD, a magnetic tape or a USB stick on which is stored electronically readable control information, in particular software (see above). All embodiments according to the invention of the method described above can be implemented when the control information is read from the data medium and stored in a controller and/or computer of a magnetic resonance apparatus.

The advantages of the magnetic resonance apparatus according to the invention and of the computer program product according to the invention essentially correspond to the advantages of the method according to the invention that are described in detail above.

### BRIEF DESCRIPTION OF THE DRAWINGS

FIG. 1 schematically illustrates a magnetic resonance apparatus according to the invention to execute a method according to the invention.

FIG. 2 shows three acquisition cycles of an acquisition sequence of an embodiment of a method according to the invention.

### DESCRIPTION OF THE PREFERRED EMBODIMENTS

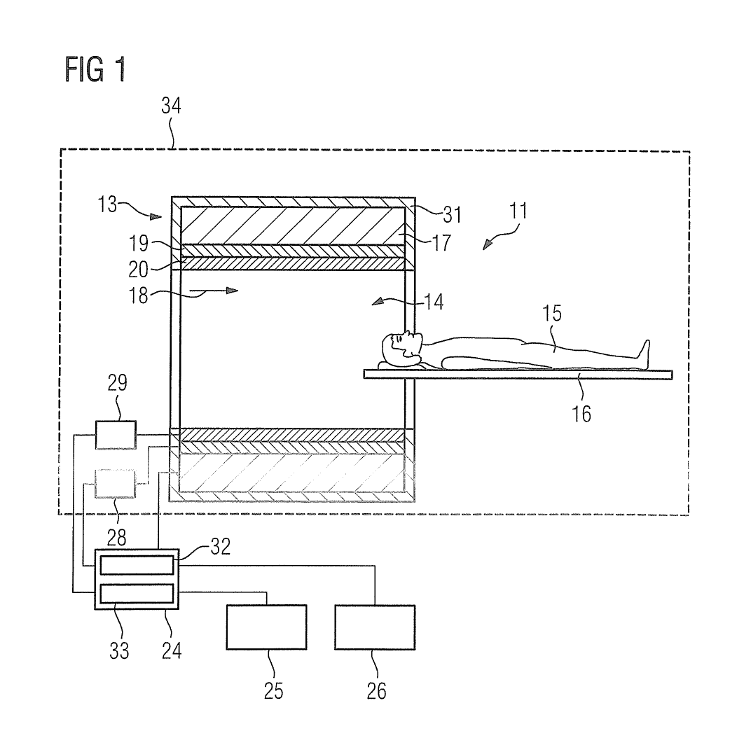

FIG. 1 shows a magnetic resonance apparatus 11 according to the invention. The magnetic resonance apparatus 11 has a detector unit (formed by a magnet unit 13) with a basic magnet 17 to generate a strong and in particular constant basic magnetic field 18. In addition to this, the magnetic resonance apparatus 11 has a cylindrical patient accommodation region 14 to accommodate an examination subject 15 (in particular a patient 15), wherein the patient accommodation region 14 is cylindrically enclosed by the magnet unit 13 in a circumferential direction. The patient 15 can be slid into the patient accommodation region 14 by means of a patient bearing device 16 of the magnetic resonance apparatus 11. For this purpose, the patient bearing device 16 has a recumbent table that is arranged so as to be movable within the magnetic resonance apparatus 11. The magnet unit 13 is externally shielded by means of a housing casing 31 of the magnetic resonance apparatus 11.

The magnet unit 13 furthermore has a gradient coil unit 19 to generate magnetic field gradients that are used for a spatial coding during an imaging. The gradient coil unit 19 is controlled by a gradient control unit 28. Furthermore, the magnet unit 13 has: a radio-frequency antenna unit 20 which, in the shown case, is designed as a body coil permanently integrated into the magnetic magnet unit 13, and a radio-frequency antenna control unit 29 to excite a polarization that arises in the basic magnetic field 18 generated by the basic magnet 17. The radio-frequency antenna unit 20 is controlled by the radio-frequency antenna control unit 29 and radiates radio-frequency magnetic resonance sequences into an examination space that is essentially formed by the patient accommodation region 14. The radio-frequency antenna unit 20 is furthermore designed to receive magnetic resonance signals, in particular from the patient 15.

The magnetic resonance apparatus 11 has a control device 24 to control the basic magnet 17, the gradient control unit 28 and the radio-frequency antenna control unit 29. The control device 24 centrally controls the magnetic resonance apparatus 11, for example the implementation of a predetermined imaging gradient echo sequence. Control information (for example imaging parameters) as well as reconstructed magnetic resonance images can be displayed to a user at a display unit 25—for example on at least one monitor—of the magnetic resonance apparatus 11. In addition, the magnetic resonance apparatus 11 has an input unit 26 that allows information and/or parameters can be input by an operator during a measurement process and/or a display process of image data. The control device 24 can include the gradient control unit 28 and/or radio-frequency antenna control unit 29 and/or the display unit 25 and/or the input unit 26.

The control device 24 has a saturation pulse generator 32 which is designed to generate a saturation pulse set with one or more saturation pulses. Furthermore, the control device 24 has a trigger module 33 which is designed to activate a first trigger window and a second trigger window on the basis of a trigger signal, wherein the first trigger window and the second trigger window are temporally delimited from one another. Furthermore, the magnetic resonance apparatus has a data acquisition device 34 which is designed for data acquisition. For example, for this the data acquisition device 34 includes the magnet unit 13, the gradient coil unit 28 and radio-frequency antenna control unit 29. For this, the saturation pulse generator 32 and the trigger module 33 can deliver control signals to the gradient control unit 28 and the radio-frequency antenna control unit 29. The magnetic resonance apparatus 11 is thus designed to execute a method according to the invention together with the control device 24.

The shown magnetic resonance apparatus 11 can naturally have additional components that magnetic resonance apparatuses 11 conventionally have. The basic functioning of a magnetic resonance apparatus 11 is known to those skilled in the art, such that a more detailed description of the additional components is not necessary herein.

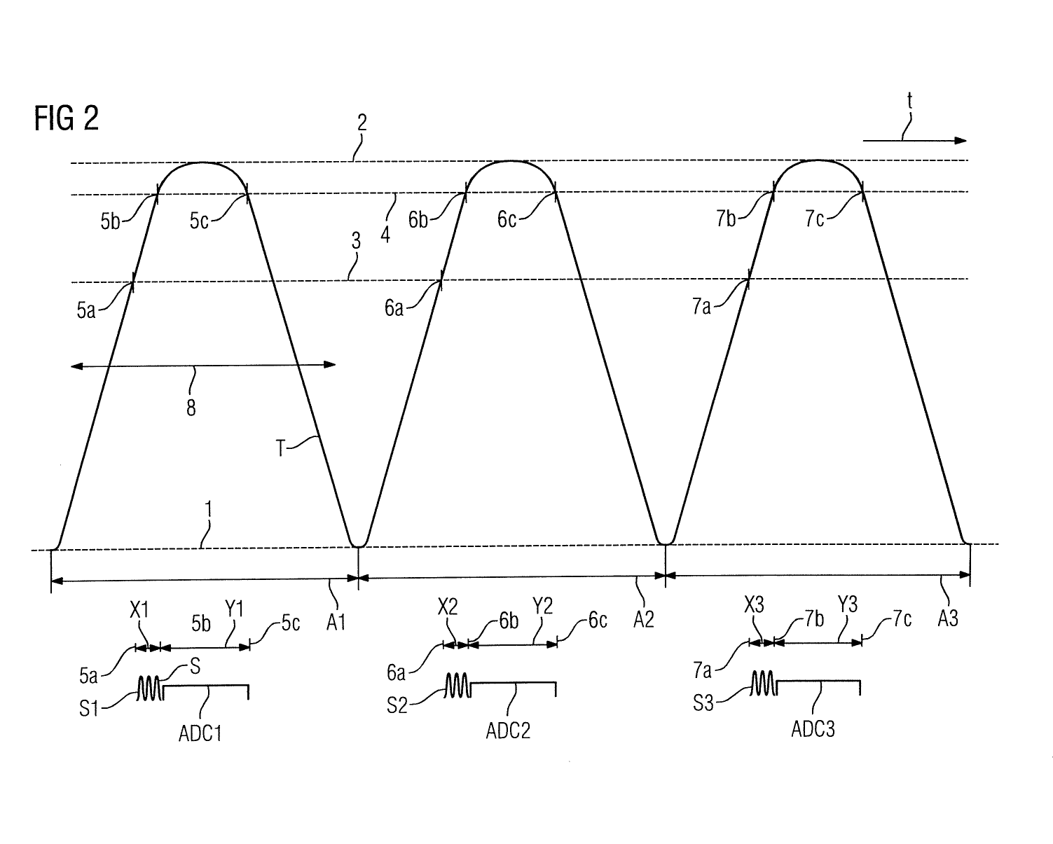

FIG. 2 shows three acquisition cycles A1, A2, A3 of an acquisition sequence of one embodiment of a method according to the invention. The acquisition sequence can naturally include additional acquisition cycles or a different number of acquisition cycles. The time curve of time t is indicated on the horizontal axis.

An acquisition cycle A1, A2, A3 thereby corresponds to a cycle of the cyclical movement of the trigger signal T. The trigger signal T is thereby a physiological signal of the patient 15, namely a signal which describes the breathing movement of the patient 15. The trigger signal T is thereby determined by means of the magnetic resonance apparatus 11 using a magnetic resonance navigator sequence. At the beginning of the acquisition sequence, a learning phase 8 to determine a pattern of the trigger signal T is thereby implemented by means of the control unit 24. The trigger signal moves between a zero position 1 which describes the maximum exhalation of the patient 15 and a maximum position 2 that describes the maximum inhalation of the patient 15. Indicated in-between these are a first threshold 3 and a second threshold 4 for the trigger signal T. As an example, the first threshold 3 thereby lies at 70 percent of the maximum position 2 of the trigger signal T. The second threshold 4 lies at 90 percent of the maximum position 2 of the trigger signal T, for example. The first threshold 3 and the second threshold 4 are thereby determined by means of the control unit 24 on the basis of the pattern of the trigger signal determined in the learning phase 8.

If, in the first acquisition cycle A1, the trigger signal T reaches the first threshold 3 at a first point in time 5a of the first acquisition cycle A1, a first trigger window X1 of the first acquisition cycle A1 is activated. If, in the first acquisition cycle A1, the trigger signal T reaches the second threshold 4 at a second point in time 5b of the first acquisition cycle A1, the first trigger window X1 of the first acquisition cycle A1 is deactivated and the second trigger window Y1 of the first acquisition cycle A1 is activated. The second trigger window Y1 of the first acquisition cycle A1 thus essentially follows immediately after the first trigger window X1 of the first acquisition cycle. However, the first trigger window X1 of the first acquisition cycle A1 and the second trigger window Y1 of the first acquisition cycle A1 are activated separately from one another on the basis of the trigger signal T. If, in the first acquisition cycle A1, the trigger signal T subsequently reaches the first threshold 3 again at a third point in time 5c of the first acquisition cycle A1, the second trigger window Y1 of the first acquisition cycle A1 is deactivated again.

The method behaves just the same in the second acquisition cycle A2 and in the third acquisition cycle A3. The second acquisition cycle A2 therefore again includes a first point in time 6a, a second point in time 6b and a third point in time 6c of the second acquisition cycle A2. These three points in time respectively establish the first trigger window X2 of the second acquisition cycle A2 and the second trigger window Y2 of the second acquisition cycle A2. Furthermore, the third acquisition cycle A3 includes a first point in time 7a, a second point in time 7b and a third point in time 7c of the third acquisition cycle A3. These three points in time respectively establish the first trigger window X3 of the third acquisition cycle A3 and the second trigger window Y3 of the third acquisition cycle A3. This scheme can repeat for additional possible acquisition cycles.

It is clear that, for all three acquisition cycles A1, A2, A3, the first trigger window X1, X2, X3 is respectively temporally delimited from the second trigger window Y1, Y2, Y3. Furthermore, it is clear that the first trigger window X1, X2, X3 and the second trigger window Y1, Y2, Y3 are respectively activated on the basis of the trigger signal T, in particular depending on the position of the trigger signal T in relation to the first threshold 3 and the second threshold 4. In each acquisition cycle A1, A2, A3, the second trigger window Y1, Y2, Y3 thereby respectively follows essentially immediately after the first trigger window X1, X2, X3. This is due to the fact that the second threshold 4 simultaneously represents the end of the first trigger window X1, X2, X3 and the start of the second trigger window Y1, Y2, Y3.

Each acquisition cycle A1, A2, A3 respectively includes a saturation pulse set S1, S2, S3 with three respective saturation pulses S. Naturally, the saturation pulse sets S1, S2, S3 can also have a deviating number of saturation pulses S. In the shown case, the saturation pulses S are designed as fat saturation pulses to saturate fat signals. The three saturation pulses S of the saturation pulse set S1, S2, S3 respectively take place during the first trigger window X1, X2, X3 of the acquisition cycles A1, A2, A3. The first threshold 3 and the second threshold 4 are chosen such that the duration of the first trigger window X1, X2, X3 respectively has a minimum size which is respectively required for the three saturation pulses S of the saturation pulse set S1, S2, S3. A data acquisition ADC1, ADC2, ADC3 respectively takes place during the second trigger window Y1, Y2, Y3 of each acquisition cycle A1, A2, A3. The data acquisition ADC1, ADC2, ADC3 can thereby respectively include additional saturation pulses S (not shown).

The function of the saturation pulses S of the saturation pulse sets S1, S2, S3 which respectively take place during the first trigger window X1, X2, X3 is again emphasized using the acquisition cycles A1, A2, A3 shown in FIG. 2. For example, if the first acquisition cycle A1 and the second acquisition cycle A2 are considered, a relatively long wait period (which, for example, is markedly longer than the duration of a data acquisition ADC1, ADC2, ADC3) elapses between the end of the data acquisition ADC1 of the first acquisition cycle A1 at the third point in time 5c of the first acquisition cycle A1 and the beginning of the data acquisition ADC2 of the second acquisition cycle A2 at the first point in time 6a of the second acquisition cycle A2. The long wait time is in particular due to the curve of the trigger signal T, thus the breathing movement of the patient 15. The data acquisition ADC1, ADC2, ADC3 takes place only during the second trigger windows Y1, Y2, Y3, each of which represent a phase of less breathing movement of the patient 15 during the inhalation of the patient 15.

If, according to conventional acquisition sequences (not shown), saturation pulses S were respectively to take place exclusively during (in particular at the beginning of) the data acquisitions ADC1, ADC2, ADC3 (thus during the second trigger window Y1, Y2, Y3), the long wait time between the acquisition cycles A1, A2, A3 would lead to an interruption of the steady state induced by the saturation pulses S. The steady state that is required for a sufficient fat saturation would thus first need to be reestablished at every data acquisition ADC1, ADC2, ADC3. This would lead to an incomplete fat saturation for the slices of the magnetic resonance images that are acquired at the beginning of the respective data acquisitions ADC1, ADC2, ADC3. The magnetic resonance images acquired by means of the conventional acquisition sequences would thus have a fat signal that varies across the slices, and thus have a low image quality.

In the case shown in FIG. 2, the pre-saturation of the fat signals because of the saturation pulses S taking place during the respective first trigger window X1, X2, X3 leads to the situation that a sufficient fat saturation is already present at the beginning of the respective data acquisition ADC1, ADC2, ADC3. For this purpose, the thresholds 3, 4 establishing the first trigger window X1, X2, X3 have been suitably matched to the second threshold 4 for the second trigger window Y1, Y2, Y3. The magnetic resonance images acquired by the acquisition sequence shown in FIG. 2 thus have a fat saturation that is homogeneously saturated across all slices, and thus have a high image quality.

The acquisition cycles of the acquisition sequence of the method according to the invention that are shown in FIG. 2 are executed by the magnetic resonance apparatus 11. For this, the magnetic resonance apparatus 11 includes required software and/or computer programs that are stored in a memory unit of the magnetic resonance apparatus 11. The software and/or computer programs include program means that are designed to execute the method according to the invention when the computer program and/or the software is executed in the magnetic resonance apparatus 11 by operation of a processor of the magnetic resonance apparatus 11. The term “processor” is not restricted to a single computing component or computer, but also encompasses distributed processing circuits or modules that operate collectively to perform the described functions.

Although modifications and changes may be suggested by those skilled in the art, it is the intention of the inventor to embody within the patent warranted hereon all changes and modifications as reasonably and properly come within the scope of his contribution to the art.

We claim as our invention:

1. A method for magnetic resonance imaging of an examination subject, comprising:
operating a magnetic resonance apparatus, in which an examination subject is situated to acquire magnetic resonance data from the examination subject in at least one data acquisition cycle, said at least one data acquisition cycle comprising radiation of a saturation pulse set, comprising at least one saturation pulse, a first trigger window, and a second trigger window;
in said at least one acquisition cycle, operating said magnetic resonance apparatus with said first trigger window and said second trigger window being temporally delimited from each other and with said first trigger window and said second trigger window being individually activated by a trigger signal and, in said first trigger window, radiating said at least one saturation pulse of said saturation pulse set and, in said second trigger window, acquiring said magnetic resonance data from said examination subject; and
entering the acquired magnetic resonance data into a memory in order to form a data file in said memory, and making said data file available as an electronic signal from said memory for further processing to form a magnetic resonance image of the examination subject.

2. A method as claimed in claim 1 comprising detecting a physiological signal from the examination subject during said acquisition cycle, and using said physiological signal as said trigger signal.

3. A method as claimed in claim 1 comprising individually activating said first trigger window dependent on an attribute of said physiological signal with respect to at least one threshold.

4. A method as claimed in claim 3 comprising, in a computerized processor, implementing a learning phase on said physiological signal to identify a pattern of said physiological signal, and determining said at least one threshold from said pattern.

5. A method as claimed in claim 3 comprising selecting said at least one threshold to cause a duration of said first trigger window to have a minimum value that is required for radiation of said at least one saturation pulse of said saturation pulse set.

6. A method as claimed in claim 1 comprising using an external signal as said trigger signal.

7. A method as claimed in claim 1 comprising, within said acquisition cycle, operating said magnetic resonance apparatus with said second trigger window following substantially immediately after said first trigger window.

8. A magnetic resonance apparatus comprising:
a magnetic resonance data acquisition unit, adapted to receive an examination subject therein, comprising a radio-frequency (RF) transmitter and a gradient system;
a computer configured to operate the magnetic resonance data acquisition unit with an examination subject situated therein to acquire magnetic resonance data from the examination subject in at least one data acquisition cycle;
said control unit, in said at least one acquisition cycle, being configured to operate said magnetic resonance apparatus with a first trigger window and a second trigger window being temporally delimited from each other by said first trigger window and said second trigger window being individually activated by a trigger signal at chronologically separated times and, in said first trigger window, radiating at least one saturation pulse of a saturation pulse set with said RF transmitter and, in said second trigger window, operating said gradient system to acquire said magnetic resonance data from said examination subject; and
an electronic memory into which the acquired magnetic resonance data by said computer, in order to form a data file in said memory, and said computer being configured to make said data file available as an electronic signal from said memory for further processing to form a magnetic resonance image of the examination subject.
